(19) United States
(12) Patent Application Publication
(10) Pub. No.: US 2025/0058046 A1
(43) Pub. Date: Feb. 20, 2025

(54) ADJUSTMENT OF AN INFUSION PUMP BASED ON PRESSURE CHANGES IN AN INFUSION LINE

(71) Applicant: CareFusion 303, Inc., San Diego, CA (US)

(72) Inventors: Richard S. WU, San Diego, CA (US); Julio Cesar Gomes PIMENTEL, San Diego, CA (US); Jay DAVE, San Diego, CA (US)

(21) Appl. No.: 18/721,178

(22) PCT Filed: Dec. 17, 2021
(86) PCT No.: PCT/US21/64230

§ 371 Date: Jun. 17, 2024

(51) Int. Cl. A61M 5/168; A61M 5/172

(52) U.S. Cl. CPC A61M 5/16854; A61M 5/172; A61M 2205/3362

(57) ABSTRACT

An infusion pump determines whether a pressure change is due to backpressure associated with a patient infusion line set associated with the intravenous infusion line or due to static pressure associated with a fluid container providing a fluid to the intravenous infusion line, determines that the pressure change did not result from an occlusion event based on the pressure change occurring over the first threshold period of time and, after determining that the pressure increase did not result from an occlusion event, the pump provides an indication that the pressure change is associated with the medical container or patient infusion line set and adjusts a motor speed of the pump or an amount of compression provided to the intravenous infusion line.

### BACKGROUND

Flow rate accuracy within infusion pump systems may be dependent on various factors, including pressure within the infusion line. Static pressure may be introduced due to the head height of the fluid container, increasing with height due to downward gravitational force. Similarly, back pressure or downstream resistance may be introduced based on the height of the infusion set above and/or below the infusion pump, on the patient side, and/or due to pressure of the patient's vascular system. Such pressures can significantly affect a pump's flow rate or a measurement of the flow rate.

### SUMMARY

The subject technology adjusts delivery volume based on back pressure and head height variations, as indicated by infusion pump pressure sensor signals. According to various aspects of the subject technology, a method for adjusting an infusion pump based on pressure changes within an infusion line comprises detecting, with one or more pressure sensors associated with an infusion pump, a pressure change outside of a pressure threshold in an intravenous infusion line associated with the infusion pump, determining whether the pressure change is due to backpressure associated with a patient infusion line set or due to static pressure associated with a medication container providing a fluid to the intravenous infusion line, determining that the pressure change occurred over a first threshold period of time, determining that the pressure change did not result from an occlusion event based on the pressure change occurring over the first threshold period of time, and after determining that the pressure change did not result from an occlusion event: when the pressure change is due to backpressure, providing an indication that the pressure change is associated with the patient infusion line set and adjusting a motor speed of the infusion pump or an amount of compression provided to the intravenous infusion line by the infusion pump based on a first algorithm; and when the pressure change is due to static pressure, providing an indication that the pressure change is associated with the medication container and adjusting the motor speed of the infusion pump or the amount of compression provided to the intravenous infusion line by the infusion pump based on a second algorithm. Other aspects include corresponding systems, apparatus, and computer program products for implementation of the corresponding method and its features.

According to various aspects of the subject technology, an infusion pump includes one or more pressure sensors, and a processor configured to: detect, with the one or more pressure sensors, a pressure change outside of a pressure threshold in an intravenous infusion line associated with the infusion pump, determine whether the pressure change is due to backpressure associated with a patient infusion line set associated with the intravenous infusion line or due to static pressure associated with a medication container providing a fluid to the intravenous infusion line, determine that the pressure change occurred over a first threshold period of time, determine that the pressure change did not result from an occlusion event based on the pressure change occurring over the first threshold period of time, and after determining that the pressure change did not result from an occlusion event: when the pressure change is due to backpressure, provide an indication that the pressure change is associated with the patient infusion line set and adjusting a motor speed of the infusion pump or an amount of compression provided to the intravenous infusion line by the infusion pump based on a first algorithm, and when the pressure change is due to static pressure, provide an indication that the pressure change is associated with the medication container and adjusting the motor speed of the infusion pump or the amount of compression provided to the intravenous infusion line by the infusion pump based on a second algorithm. Other aspects include corresponding systems, methods, and computer program products for implementation of the corresponding infusion pump.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description below, in conjunction with the following drawings. Like reference numerals refer to corresponding parts throughout the figures and description.

FIG. 1 depicts an example infusion pump set-up, shown in use in its intended environment.

FIG. 2, an infusion pump is shown in perspective view with the front door open, showing the upstream fluid line and downstream fluid line in operative engagement with the pump.

FIGS. 3A and 3B depict example processes for adjusting an infusion pump based on pressure changes within an infusion line.

FIGS. 4A and 4B depict example guard bands for determining whether a pressure change within an infusion line indicates an adjustment to infusion pump should be made.

FIG. 5A depicts an example process for adjusting an infusion pump based on a pressure change feedback loop. FIG. 5B depicts an example process for adjusting an infusion pump based on training or manufacturer data.

FIG. 6 is a conceptual diagram illustrating an example electronic system for adjusting an infusion pump based on pressure changes in an infusion line.

### DETAILED DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

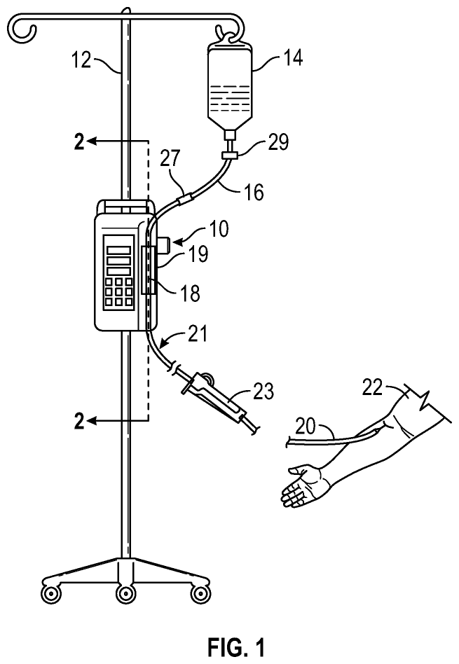

FIG. 1 depicts an example infusion pump set-up 10, shown in use in its intended environment, according to various aspects of the subject technology. In particular, the infusion pump set-up 10 is shown mounted to an intravenous (IV) pole 12 on which a fluid source 14 containing an IV fluid is held. The fluid source 14 is connected in fluid communication with an upstream fluid line 16. The fluid line 21 is a conventional IV infusion type tube typically used in a hospital or medical environment, and is made of any type of flexible tubing appropriate for use to infuse therapeutic fluids into a patient, such as polyvinylchloride (PVC). A flexible pumping fluid line portion 18 is mounted in operative engagement with a peristaltic pumping apparatus 19, for propelling fluid through a downstream fluid line portion 20, for example, to a patient's arm 22.

It will be understood by those skilled in the art that the upstream fluid line portion 16, the pumping flexible line portion 18, and the downstream fluid line portion 20 may be portions of a continuous length of a flexible tubing, with the portions defined by the location of the peristaltic pump 19, or detachable sections that, when coupled, form a continuous infusion line. For convenience, the continuous length of the flexible tubing of the infusion line is indicated by numeral 21. A roller clamp 23 (e.g., configured to provide for mechanical compression of the line to block the flow) may be positioned on the downstream fluid line 20 between the pump 10 and the patient's arm 22. In this context, the term “upstream” refers to that portion of the flexible tubing that extends between the fluid source and peristaltic pump, and the term “downstream” refers to that portion of the flexible tubing that extends from the peristaltic pump to the patient.

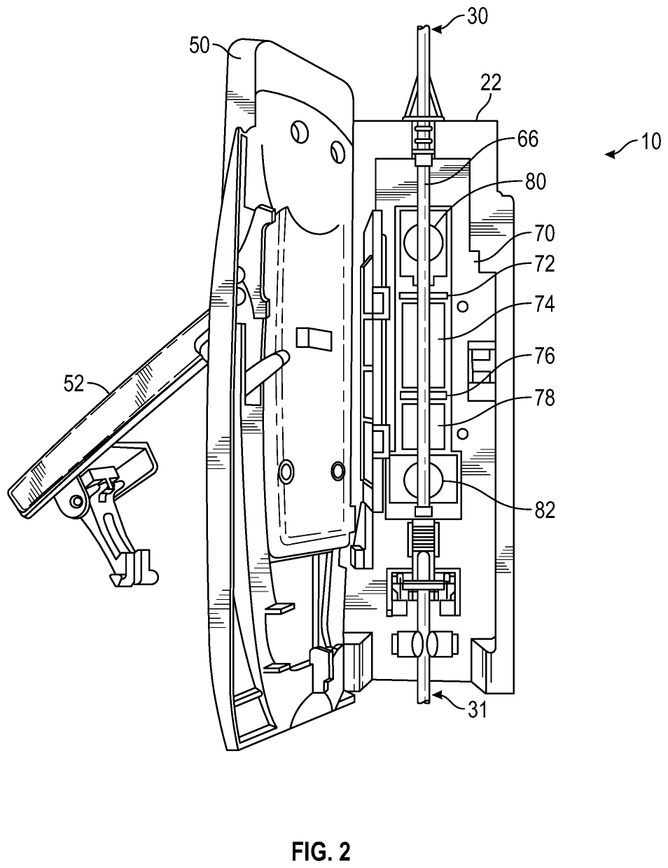

Turning now to FIG. 2, an infusion pump 10 is shown in perspective view with the front door 50 open, showing the upstream fluid line 30 and downstream fluid line 31 in operative engagement with the pump 10. The infusion pump 10 directly acts on a tube 66 that connects the upstream fluid line 30 to the downstream fluid line 31 to form a continuous fluid conduit, extending from the respective fluid supply 14 and/or 25 (FIG. 1) to the patient 22, 31, through which fluid is acted upon by the pump to move fluid downstream to the patient. Specifically, a pumping mechanism 70 acts as the flow control device of the pump to move fluid though the conduit. The upstream and downstream fluid lines and/or tube 66 may be coupled to a pump cassette or cartridge that is configured to be coupled to the pump 10.

The type of pumping mechanism may vary and may be for example, a multiple finger pumping mechanism. For example, the pumping mechanism may be of the “four finger” type and includes an upstream occluding finger 72, a primary pumping finger 74, a downstream occluding finger 76, and a secondary pumping finger 78. The “four finger” pumping mechanism and mechanisms used in other linear peristaltic pumps operate by sequentially pressing on a segment of the fluid conduit by means of the cam-following pumping fingers and valve fingers 72, 74, 76, and 78. A dynamically controllable motor may be included to control the pumping fingers based at least in part on the infusion line pressure monitoring features described. The pressure is applied in sequential locations of the conduit, beginning at the upstream end of the pumping mechanism and working toward the downstream end. At least one finger is always pressing hard enough to occlude the conduit. As a practical matter, one finger does not retract from occluding the tubing until the next one in sequence has already occluded the tubing; thus, at no time is there a direct fluid path from the fluid supply to the patient. The operation of peristaltic pumps including four finger pumps is well known to those skilled in the art and no further operational details are provided here.

In this particular embodiment, FIG. 2 further shows a downstream pressure sensor 82 included in the pump 10 embodiment at a downstream location with respect to the pumping mechanism. The downstream pressure sensor 82 is mounted to the flow control device 70 and is located adjacent and downstream in relation to the flow control device. The downstream pressure sensor is located downstream from the flow control device, that is, at a location between the patient 22 (FIG. 1) and the flow control device, so that the connection of the correct fluid supply with the correct pump may be verified before any fluid is pumped to the patient.

With reference still to FIG. 2, an upstream pressure sensor 80 may also be included in the pump 10. The upstream pressure sensor is assigned to the flow control device or pumping mechanism 70 and, in this example, is further provided as an integral part of the pump 10. It is mounted to the flow control device 70 and is located adjacent and upstream in relation to the flow control device. The upstream pressure sensor is located upstream from the flow control device, that is, at a location between the fluid supply 14 and/or 25 (FIG. 1) and the flow control device, so that the connection of the correct fluid supply with the correct pump may be verified before any fluid is pumped to the patient. The elements shown in FIGS. 1 and 2 may be adjusted to increase efficiency of resource utilization and/or safe operation of the pump using one or more the pressure change detection features described.

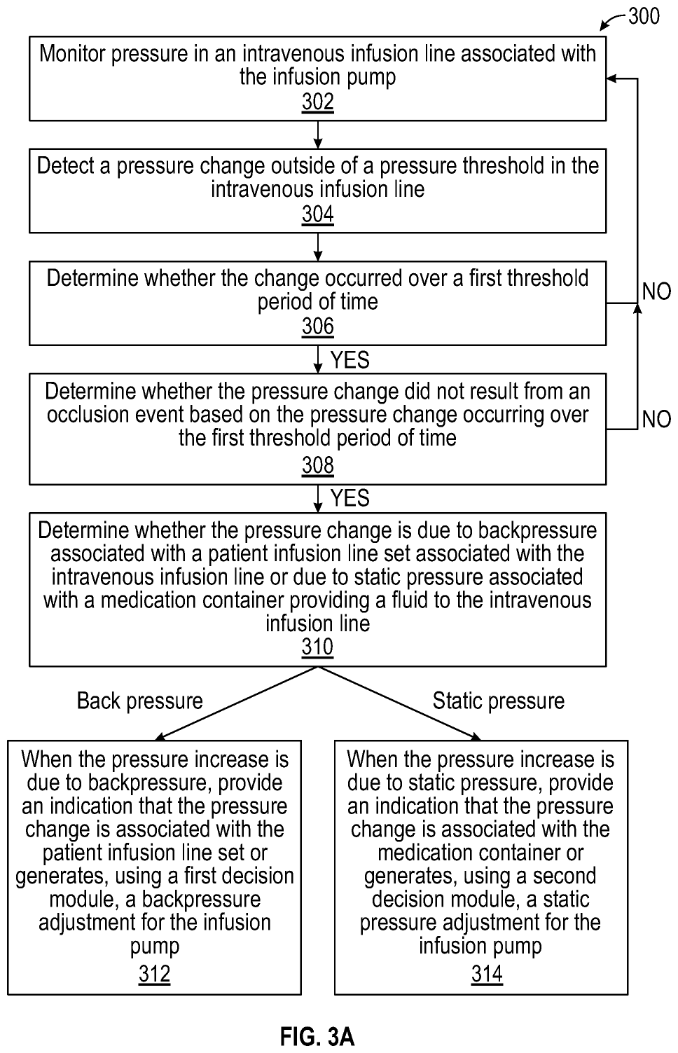

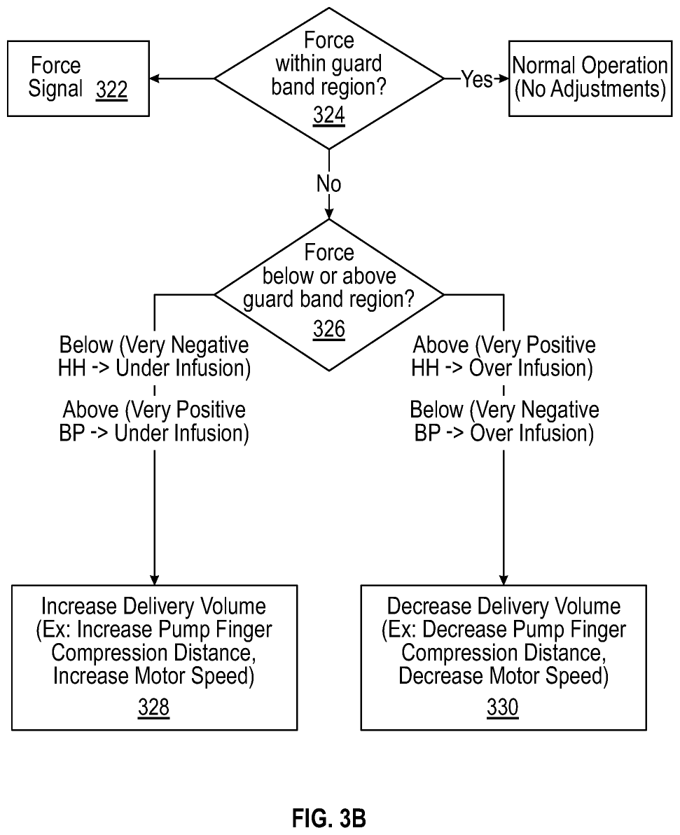

FIG. 3A depicts a first example process 300 for adjusting an infusion pump based on pressure changes within an infusion line, according to aspects of the subject technology. For explanatory purposes, the various blocks of example process 300 are described herein with reference to FIGS. 1 and 2, and the components and/or processes described herein. The one or more of the blocks of process 300 may be implemented, for example, by one or more computing devices including, for example, pump 10. In some implementations, one or more of the blocks may be implemented based on one or more machine learning algorithms. In some implementations, one or more of the blocks may be implemented apart from other blocks, and by one or more different processors or devices. Further for explanatory purposes, the blocks of example process 300 are described as occurring in serial, or linearly. However, multiple blocks of example process 300 may occur in parallel. In addition, the blocks of example process 300 need not be performed in the order shown and/or one or more of the blocks of example process 300 need not be performed.

While pump 10 is described as implementing the steps of process 300, it is understood that the steps of process 300 may also or alternatively be implemented and/or facilitated by one or more processors within pump 10, or within a system associated with pump 10 more generally. For example, the steps of process 300 may be implemented by a remote computing device or other device(s) based on data received from pressure sensors 80, 82 of pump 10 or from pressure sensors associated with the infusion line 21 at locations outside the pump 10, such as located at upstream infusion line 16 and/or at downstream infusion line 20.

In the depicted example, pump 10 monitors pressure in an intravenous infusion line 21 using upstream pressure sensor 80 and downstream pressure sensor 82 (302). According to various implementations, the pump 10 detects, with one or more of the pressure sensors 80, 82, a pressure change outside of a pressure threshold in the intravenous infusion line 21 (304). For example, upstream pressure sensor 80 may be used to detect pressure within the upstream infusion line portion 16 or, in some cases, portions 18 or 20; and downstream pressure sensor 82 may be used to detect pressure within the downstream infusion line portion 20 or, in some cases, portions 16 or 18. The pressure threshold may be a predetermined expected threshold pressure programmed into the pump, or an expected pressure range.

When a pressure change is detected, process 300 determines whether the change occurred over a first threshold period of time (306). When the pressure change occurred over the threshold period, the pump 10 can determine that the pressure change did not result from an occlusion event (308).

In the depicted example, the pump 10 determines whether the pressure change is due to backpressure associated with a back pressure from a patient infusion line set 20 associated with the intravenous infusion line 21 or due to static pressure associated with a medication container 14 that provides a fluid to the intravenous infusion line 21 (310). In some implementations, this determination may include comparing a pressure change detected using the upstream sensor 80 and a pressure change detected using the downstream sensor 82. The weighted difference may be indicative of whether the pressure change is primarily a result of static pressure or line resistance or back pressure. In some implementations, each change is determined and then summed, and the net difference (e.g., whether positive or negative) may determine whether the pressure change is primarily a result of static pressure or line resistance or back pressure. For example, a net positive difference may be indicative of a static pressure due to head height of a medication container 14 and a net negative difference may be indicative of a back pressure from the patient infusion set 20, or vice versa.

After the pump 10 determines that the pressure increase did not result from an occlusion event, the pump 10 takes different actions depending on whether the pressure change is due to back pressure or static pressure. When the pressure increase is due to backpressure, the pump 10 provides an indication that the pressure change is associated with the patient infusion line set or generates, using a first decision model, a backpressure adjustment for the infusion pump (312). According to some implementations, the first decision model is configured to generate the backpressure adjustment based at least in part on the pressure change and a characteristic of the infusion pump during the first threshold period of time, and cause the pump 10 to adjust an operation in accordance with the backpressure adjustment. In some implementations, adjusting the operation includes adjusting a motor speed of the pump 10 or an amount of compression provided to the intravenous infusion line by the infusion pump based on a first algorithm. According to various implementations, providing the indication that the pressure change is associated with the patient infusion line set may include providing an audio or visual indication that a height of the patient infusion line set with respect to the infusion pump should be adjusted. The amount of compression provided to the intravenous line may be adjusted, for example, by adjusting a pump finger compression distance.

When the pressure increase is due to static pressure, the pump 10 provides an indication that the pressure change is associated with the medication container or generates, using a second decision model, a static pressure adjustment for the infusion pump (314). According to some implementations, the second decision model is configured to generate the static adjustment based at least in part on the pressure change, a first characteristic of the infusion pump during the first threshold period of time, and a second characteristic of the fluid container (e.g., a current amount of fluid remaining in the container, height of the container, etc.), and cause the pump 10 to adjust an operation in accordance with the static pressure adjustment. In some implementations, adjusting the operation includes adjusting the motor speed of the pump or the amount of compression provided to the intravenous infusion line by the infusion pump based on a second algorithm (314). According to various implementations, providing the indication that the pressure change is associated with the medication container may include providing an audio or visual indication that a height of the medication container with respect to the infusion pump should be adjusted.

According to various implementations, the first and/or second algorithms may be selected based on whether the pressure change is predominately due to backpressure or static pressure. The first algorithm and/or second algorithm may, for example, involve a different magnitude multiplier for determining the delivery volume adjustment based on the determined pressure change. In some implementations, each algorithm may be supported by a lookup table that indexes an adjustment based on the pressure change or use closed loop feedback. According to various implementations, the motor speed of the pump 10 may be adjusted to change the delivery volume, or the amount of compression provided to the intravenous infusion line, based on the first and second algorithms. For example, the result of each algorithm may be summed to provide an adjusted motor speed or amount of compression.

Motor speed can be increased or decreased to increase or decrease (respectively) the delivery volume to help compensate for under infusion or over infusion (respectively) caused by the pressure change due to backpressure or static pressure. The amount of compression provided to the intravenous infusion line can be increased or decreased to increase or decrease (respectively) the delivery volume to help compensate for under infusion or over infusion (respectively) caused by the pressure change due to backpressure or static pressure. The specific amount of motor speed increase/decrease and/or specific amount of compression increase/decrease can be based on a lookup table or using closed loop feedback.

For example, the process 300 may include generating, using a first decision model, a backpressure adjustment for the infusion pump upon detecting a pressure change due to backpressure. The model may generate the backpressure adjustment using the pressure change and a characteristic of the infusion pump during the first time period (e.g., programmed parameter, infusion set, pump type, etc.). The adjustment may be used to cause the infusion pump to adjust operation to address the backpressure.

In cases where static pressure is detected, the process 300 may include generating, using a second decision model, a static pressure adjustment for the infusion pump upon detecting a pressure change due to static pressure. The model may generate the static pressure adjustment using the pressure change, a first characteristic of the fluid container (e.g., head height, size, etc.) during the first time period, and the characteristic pump during the first time period (e.g., programmed parameter, infusion set, pump type, etc.). The adjustment may be used to cause the infusion pump to adjust operation to address the static pressure change.

As used herein, a “decision model” may include a function or machine learning model configured to receive, as inputs, a pressure change and other characteristics of the infusion pump such as an identifier for the infusion pump, an identifier for the administration set, identifier of the container, height of the container, or operational parameters of the infusion pump such as motor speed, rate of infusion, or the like, and provide, as an output, an adjustment to the in infusion pump that is likely to address the backpressure. Example adjustments include motor speed adjustment, compression applied to the administration set by the infusion pump, audio or visual indicators to change the user interface or other output device associated with the infusion pump, or the like.

Many of the above-described example process 300, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 3B depicts a second example process 320 for adjusting an infusion pump based on pressure changes within an infusion line, according to aspects of the subject technology. For explanatory purposes, the various blocks of example process 320 are described herein with reference to FIGS. 1 and 2, and the components and/or processes described herein. The one or more of the blocks of process 320 may be implemented, for example, by one or more computing devices including, for example, pump 10. In some implementations, one or more of the blocks may be implemented based on one or more machine learning algorithms. In some implementations, one or more of the blocks may be implemented apart from other blocks, and by one or more different processors or devices. Further for explanatory purposes, the blocks of example process 320 are described as occurring in serial, or linearly. However, multiple blocks of example process 320 may occur in parallel. In addition, the blocks of example process 320 need not be performed in the order shown and/or one or more of the blocks of example process 320 need not be performed.

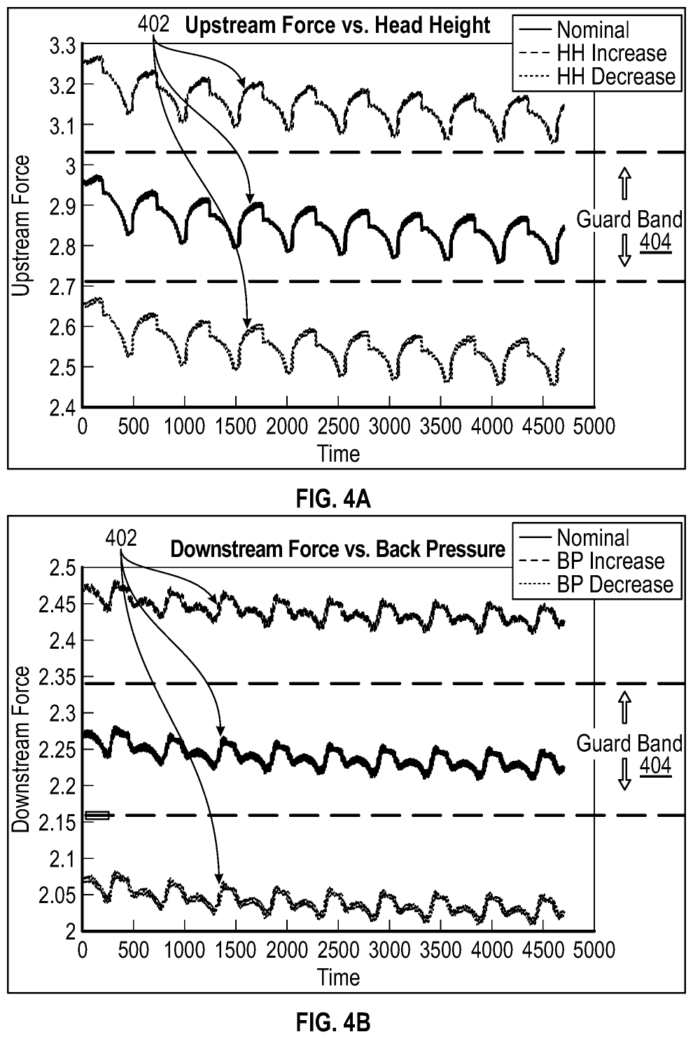

As shown in FIG. 3B, each of the upstream pressure sensor 80 and downstream sensor 82 provide a pressure signal that is monitored. In the depicted example, a pressure signal is received (322) and the magnitude of the pressure signal is compared with a predetermined guard band (324), as shown further in FIGS. 4A and 4B. Based on the magnitude being below or above the band region (326), the delivery volume (or flow rate) of the pump 10 may be increased (328) or decreased (330). In some implementations, if the pressure signal from the upper pressure sensor 80 is below the guard band region, or the pressure signal from the downstream pressure sensor 82 is above the guard band region, then an under infusion may be indicated and the delivery volume (or flow rate) may be increased. If the pressure signal from the upper pressure sensor 80 is above the guard band region, or the pressure signal from the downstream pressure sensor 82 is below the guard band region, then an over infusion may be indicated and the delivery volume (or flow rate) may be decreased.

FIGS. 4A and 4B depict example guard bands for determining whether a pressure change within an infusion line indicates an adjustment to infusion pump should be made, according to aspects of the subject technology. FIG. 4A depicts an example guard band and related electrical signals corresponding to pressure changes associated with a head height of a medication container 14. FIG. 4B depicts an example guard band and related electrical signals corresponding to pressure changes associated with a back pressure of an infusion set 20.

As shown in the depicted examples, a pressure signal 402 may be time varying, changing in conjunction with the pump's compression of the infusion tubing 21. For example, the signals 402 may represent a pumping cycle associated with a cyclical pumping operation of the pump to move the fluid through the intravenous infusion line 21. Generally, a nominal signal will remain within a predetermined guard band range, as depicted. When the signal is above or below the guard band then the pump 10 (or system) may determine that an adjustment of the pump's motor, compression amount, or compression rate should be made, as described previously.

It has been found that relaxation of the flexible tubing 21 may not have any significant effect on actual pressure but can have a significant effect on pressure measured by the pressure sensors 80, 82. Accordingly, the pump 10 may undertake an analysis of the electrical signals to determine (based on the signal values) whether the pressure change results from relaxation of the intravenous infusion line. The motor speed or amount or rate of compression may then be adjusted only after the pressure change is determine to not result from relaxation of the intravenous infusion line.

Because relaxation of the infusion tubing may occur over time, the pump 10 may not determine that a signal outside of the guard band is out of tolerance until the signal 402 is outside for a predetermined period of time. Additionally or in the alternative, because relaxation typically occurs over time, the overall magnitude and/or slope of signal itself may change over time, as depicted in FIGS. 4A and 4B, which show the signals steadily decreasing over about 4500 seconds.

The pump 10 may determine a standardized signal level corresponding to the time-varying signals, such as a DC voltage level, a mean or average voltage level. In this regard, detecting the pressure change is outside of the pressure threshold may include detecting that the standardized signal level is outside the pressure threshold for the threshold period of time.

Since the standardized signal may steadily decrease due to relaxation, as depicted in FIGS. 4A and 4B, the pump 10 may also change the guard bands 404 to account for the relaxation. Accordingly, the pump 10 may determine a decay of the standardized signal level and adjust the pressure threshold (of the guard band(s)) to correspond to the decay. For example, if the standardized signal is found to have a logarithmic decay then the logarithmic decay may be applied to the guard bands, such that each pressure threshold of the guard bands decays logarithmically over time. The standardized signal level of the pressure signal(s) may then be compared to the adjusted pressure threshold. If the standardized signal is determined to not fall outside the adjusted pressure threshold then the pressure change requiring adjustment may be determined not to be a function of relaxation.

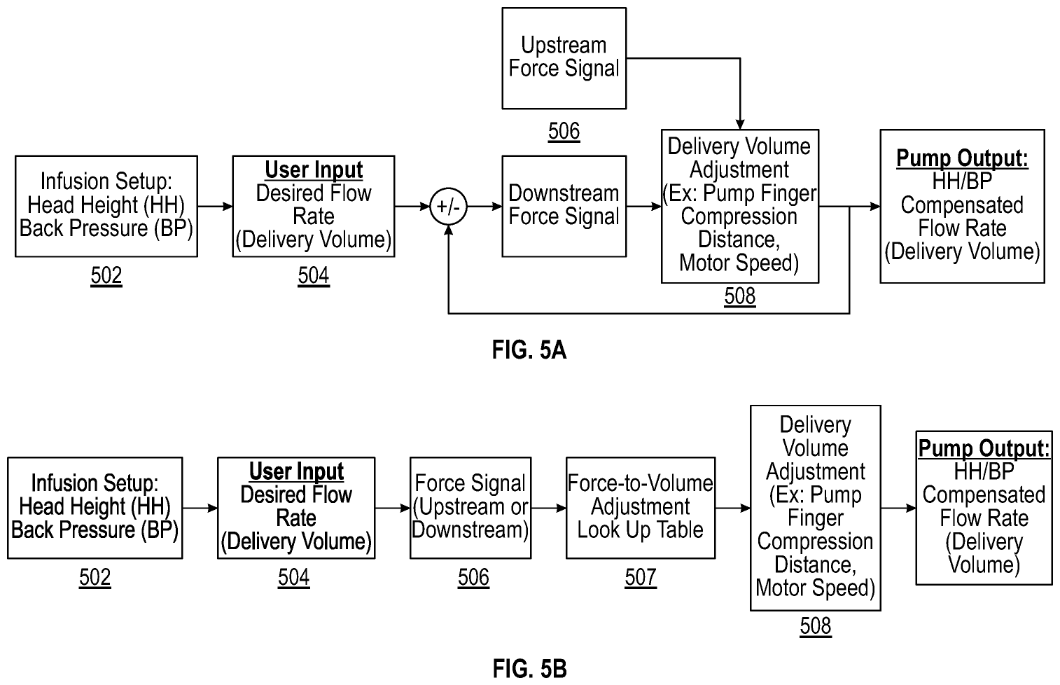

FIG. 5A depicts an example process for adjusting an infusion pump based on a pressure change feedback loop, according to aspects of the subject technology. In the depicted example, a clinician sets up the pump 10 system, including positioning and connecting the medication container 14 and infusion set 20, and loading the infusion tubing 21 within the pump (502). The clinician then inputs various parameters into the pump 10 to start an infusion, including a desired flow rate (504).

In the depicted example, the pressure signals from the upstream pressure sensor 80 and the downstream pressure sensor 82 are measured (506) and the delivery volume adjusted (508), as described in FIGS. 3A and/or 3B. The adjustment amount may be a predetermined amount intended to incrementally adjust the pumping mechanisms so that the magnitude of the measured pressure change is adjusted toward being within the guard bands, or achieve the predetermined desired flow rate. In the later case, the pump 10 may be connected to a flow rate detector, obtain flow rate from the detector, and iteratively adjust the pumping mechanisms until the obtained flow rate is within a tolerance of the predetermined desired flow rate.

After the motor speed of the infusion pump or the amount of compression provided to the intravenous infusion line is adjusted, the pump 10 may query the downstream pressure sensor 82 of the infusion pump 10 to determine whether the pressure change remains outside of the pressure threshold. If the pressure change remains outside of the threshold then the pump may iteratively adjust, based on a magnitude of the downstream pressure signal, the motor speed of the infusion pump or the amount of compression provided to the intravenous infusion line to cause the pressure change to be within the pressure threshold. In this manner, the adjustment may be a predetermined iterative amount to compensate for the out of tolerance pressure, and the pump may repeat the monitoring and adjustments until the pressure within the infusion line 21 is no longer outside of the pressure threshold. In some implementations, parameters pertaining to a desired flow rate and a type of the intravenous infusion line 21 may be obtained by the pump 10 before (or after) the pressure change is detected, and the motor speed of the pump or the amount of compression provided to the intravenous infusion line the adjusted to adjust a current flow rate of the fluid within the intravenous infusion line towards the desired flow rate.

FIG. 5B depicts an example process for adjusting an infusion pump based on training or manufacturer data, according to aspects of the subject technology. In the depicted example, a clinician sets up the pump 10 system, including positioning and connecting the medication container 14 and infusion set 20, and loading the infusion tubing 21 within the pump (502). The clinician then inputs various parameters into the pump 10 to start an infusion, including a desired flow rate (504). The pressure signals from the upstream pressure sensor 80 and the downstream pressure sensor 82 are measured (506). In the depicted example, however, the adjustment is determined based on a lookup table (507), for example, based on the magnitude of the signal(s) and/or deviation from the threshold. The delivery volume is then adjusted (508), as described in FIGS. 3A and/or 3B with the value retrieved from the lookup table. The adjustment amount may be a predetermined amount indexed by the measured flow rate, the deviation of the measured flow rate from the desired flow rate, deviation of the pressure signal from the guard band(s).

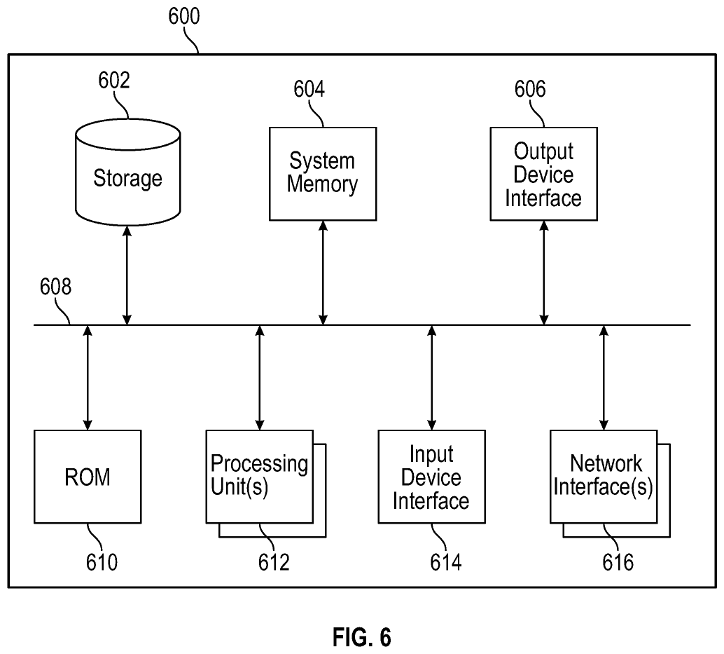

FIG. 6 is a conceptual diagram illustrating an example electronic system 600 for adjusting an infusion pump based on pressure changes in an infusion line, according to aspects of the subject technology. Electronic system 600 may be a computing device for execution of software associated with one or more portions or steps of method 500, or components and methods provided by FIGS. 1-5, including but not limited to computing hardware within patient care device 12, or syringe pump 200, and/or any computing devices or associated terminals disclosed herein. In this regard, electronic system 600 may include the infusion pump 10, a computing device within or connected to the infusion pump 10.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and methods previously described.

Bus 608 collectively represents all system, peripheral, and chipset buses that communicatively connect the numerous internal devices of electronic system 600. For instance, bus 408 communicatively connects processing unit(s) 612 with ROM 610, system memory 604, and permanent storage device 602.

From these various memory units, processing unit(s) 612 retrieves instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process, in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 600 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software, depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method comprising: detecting, with one or more pressure sensors associated with an infusion pump, a pressure change outside of a pressure threshold in an intravenous infusion line associated with the infusion pump; determining whether the pressure change is due to backpressure associated with a infusion line set connected to the intravenous infusion line or due to static pressure associated with a fluid container providing a fluid to the intravenous infusion line, said determining based at least in part on which of the one or more pressure sensors associated with the infusion pump detected the pressure chance; determining that the pressure change occurred over a first threshold period of time; determining that the pressure change did not result from an occlusion event based on the pressure change occurring over the first threshold period of time; and after determining that the pressure change did not result from an occlusion event: when the pressure change is due to backpressure, the method includes at least one of: (a) providing a first indication that the pressure change is associated with the patient infusion line set, and (b) generating, using a first decision model, a backpressure adjustment for the infusion pump, the first decision model configured to generate the backpressure adjustment based at least in part on the pressure change and a characteristic of the infusion pump during the first threshold period of time, and causing the infusion pump to adjust operation in accordance with the backpressure adjustment; and when the pressure change is due to static pressure, the method includes at least one of: providing a second indication that the pressure change is associated with the fluid container, and generating, using a second decision model, a static pressure adjustment for the infusion pump, the second decision model configured to generate the static pressure adjustment based at least in part on the pressure change, a first characteristic of the infusion pump during the first threshold period of time, and a second characteristic of the fluid container, and causing the infusion pump to adjust operation in accordance with the static pressure adjustment.

Clause 2. The method of Clause 1, wherein providing the indication that the pressure change is associated with the fluid container comprises providing an audio or visual indication that a height of the fluid container with respect to the infusion pump should be adjusted.

Clause 3. The method of any one of Clauses 1 or 2, wherein providing the indication that the pressure change is associated with the patient infusion line set comprises providing an audio or visual indication that a height of the patient infusion line set with respect to the infusion pump should be adjusted.

Clause 4. The method of any one of Clauses 1 through 3, further comprising: analyzing electrical signals representative of a pumping cycle associated with a cyclical pumping operation of the pump to move the fluid through the intravenous infusion line; determining, based on the electrical signals, that that the pressure change did not result from relaxation of the intravenous infusion line, wherein the motor speed or amount of compression is adjusted after the pressure change is determine to not result from relaxation of the intravenous infusion line.

Clause 5. The method of Clause 4, wherein the electrical signals comprise time-varying signal levels, and wherein analyzing the electrical signals comprises: determining a standardized signal level corresponding to the time-varying signal levels, wherein detecting the pressure change is outside of the pressure threshold comprises detecting the standardized signal level is outside the pressure threshold for a second threshold period of time.

Clause 6. The method of Clause 5, wherein determining that that the pressure change did not result from relaxation of the intravenous infusion line comprises: determining a decay of the standardized signal level; adjusting the pressure threshold to correspond to the decay; comparing the standardized signal level to the adjusted pressure threshold; and confirming that the standardized signal level is outside the adjusted pressure threshold for a second period of time.

Clause 7. The method of any one of Clauses 1 through 6, wherein causing the infusion pump to adjust operation in accordance with the backpressure adjustment comprises adjusting a motor speed of the infusion pump or an amount of compression provided to the intravenous infusion line by the infusion pump based on a first algorithm, and wherein causing the infusion pump to adjust operation in accordance with the static pressure adjustment comprises adjusting a motor speed of the infusion pump or an amount of compression provided to the intravenous infusion line by the infusion pump based on a second algorithm.

Clause 8. The method any one of Clauses 1 through 7, after adjusting the motor speed of the infusion pump or the amount of compression provided to the intravenous infusion line: obtaining, from a downstream pressure sensor of the infusion pump, a downstream pressure signal indicating that the pressure change remains outside of a pressure threshold; and iteratively adjusting, based on a magnitude of the downstream pressure signal, the motor speed of the infusion pump or the amount of compression provided to the intravenous infusion line to cause the pressure change to be within the pressure threshold.

Clause 9. The method of any one of Clauses 1 through 8, wherein the pressure change is due to static pressure and the amount of compression provided to the intravenous infusion line is adjusted by adjusting a pump finger compression distance.

Clause 10. The method of Clause any one of Clauses 1 through 9, further comprising: obtaining, by the infusion pump before the pressure change is detected, parameters pertaining to a desired flow rate and a type of the intravenous infusion line; wherein the motor speed of the infusion pump or an amount of compression provided to the intravenous infusion line is adjusted to adjust a current flow rate of the fluid within the intravenous infusion line towards the desired flow rate.

Clause 11. An infusion pump, comprising: one or more pressure sensors; a processor configured to: detect, with the one or more pressure sensors, a pressure change outside of a pressure threshold in an intravenous infusion line associated with the infusion pump; determine whether the pressure change is due to backpressure associated with a patient infusion line set associated with the intravenous infusion line or due to static pressure associated with a fluid container providing a fluid to the intravenous infusion line; determine that the pressure change occurred over a first threshold period of time; determine that the pressure change did not result from an occlusion event based on the pressure change occurring over the first threshold period of time; and after determining that the pressure change did not result from an occlusion event: when the pressure change is due to backpressure, provide an indication that the pressure change is associated with the patient infusion line set and adjusting a motor speed of the infusion pump or an amount of compression provided to the intravenous infusion line by the infusion pump based on a first algorithm; and when the pressure change is due to static pressure, provide an indication that the pressure change is associated with the fluid container and adjusting the motor speed of the infusion pump or the amount of compression provided to the intravenous infusion line by the infusion pump based on a second algorithm.

Clause 12. The infusion pump of Clause 11, wherein providing the indication that the pressure change is associated with the fluid container comprises providing an audio or visual indication that a height of the fluid container with respect to the infusion pump should be adjusted.

Clause 13. The infusion pump of Clause 11 or 12, wherein providing the indication that the pressure change is associated with the patient infusion line set comprises providing an audio or visual indication that a height of the patient infusion line set with respect to the infusion pump should be adjusted.

Clause 14. The infusion pump of any one of Clauses 11 through 13, wherein the processor is further configured to: analyze electrical signals representative of a pumping cycle associated with a cyclical pumping operation of the pump to move the fluid through the intravenous infusion line; determine, based on the electrical signals, that that the pressure change did not result from relaxation of the intravenous infusion line, wherein the motor speed or amount of compression is adjusted after the pressure change is determine to not result from relaxation of the intravenous infusion line.

Clause 15. The infusion pump of Clause 14, wherein the electrical signals comprise time-varying signal levels, and wherein analyzing the electrical signals comprises: determining a standardized signal level corresponding to the time-varying signal levels, wherein detecting the pressure change is outside of the pressure threshold comprises detecting the standardized signal level is outside the pressure threshold for a second threshold period of time.

Clause 16. The infusion pump of Clause 15, wherein determining that that the pressure change did not result from relaxation of the intravenous infusion line comprises: determining a decay of the standardized signal level; adjusting the pressure threshold to correspond to the decay; comparing the standardized signal level to the adjusted pressure threshold; and confirming that the standardized signal level is outside the adjusted pressure threshold for a second period of time.

Clause 17. The infusion pump of any one of Clauses 11 through 16, wherein the processor is further configured to: determine that the pressure change is due to backpressure and static pressure; adjust the motor speed of the infusion pump or the amount of compression provided to the intravenous infusion line based on the first and second algorithms.

Clause 18. The infusion pump of any one of Clauses 1 through 17, wherein the processor is further configured to, after adjusting the motor speed of the infusion pump or the amount of compression provided to the intravenous infusion line: obtain, from a downstream pressure sensor of the infusion pump, a downstream pressure signal indicating that the pressure change remains outside of a pressure threshold; and iteratively adjust, based on a magnitude of the downstream pressure signal, the motor speed of the infusion pump or the amount of compression provided to the intravenous infusion line to cause the pressure change to be within the pressure threshold.

Clause 19. The infusion pump of Clause 18, wherein the pressure change is due to static pressure and the amount of compression provided to the intravenous infusion line is adjusted by adjusting a pump finger compression distance.

Clause 20. The infusion pump of any one of Clauses 11 through 19, wherein the processor is further configured to: obtain, by the infusion pump before the pressure change is detected, parameters pertaining to a desired flow rate and a type of the intravenous infusion line, wherein the motor speed of the infusion pump or an amount of compression provided to the intravenous infusion line is adjusted to adjust a current flow rate of the fluid within the intravenous infusion line towards the desired flow rate.

### Further Consideration

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine-readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In some implementations, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. A method comprising:
detecting, with one or more pressure sensors associated with an infusion pump, a pressure change outside of a pressure threshold in an intravenous infusion line associated with the infusion pump;
determining whether the pressure change is due to backpressure associated with a infusion line set connected to the intravenous infusion line or due to static pressure associated with a fluid container providing a fluid to the intravenous infusion line, said determining based at least in part on which of the one or more pressure sensors associated with the infusion pump detected the pressure chance;
determining that the pressure change occurred over a first threshold period of time;
determining that the pressure change did not result from an occlusion event based on the pressure change occurring over the first threshold period of time; and
after determining that the pressure change did not result from an occlusion event:
when the pressure change is due to backpressure, the method includes at least one of:
(a) providing a first indication that the pressure change is associated with the patient infusion line set, and
(b) generating, using a first decision model, a backpressure adjustment for the infusion pump, the first decision model configured to generate the backpressure adjustment based at least in part on the pressure change and a characteristic of the infusion pump during the first threshold period of time, and causing the infusion pump to adjust operation in accordance with the backpressure adjustment; and
when the pressure change is due to static pressure, the method includes at least one of:
(a) providing a second indication that the pressure change is associated with the fluid container, and
(b) generating, using a second decision model, a static pressure adjustment for the infusion pump, the second decision model configured to generate the static pressure adjustment based at least in part on the pressure change, a first characteristic of the infusion pump during the first threshold period of time, and a second characteristic of the fluid container, and causing the infusion pump to adjust operation in accordance with the static pressure adjustment.

2. The method of claim 1, wherein providing the indication that the pressure change is associated with the fluid container comprises providing an audio or visual indication that a height of the fluid container with respect to the infusion pump should be adjusted.

3. The method of claim 1, wherein providing the indication that the pressure change is associated with the patient infusion line set comprises providing an audio or visual indication that a height of the patient infusion line set with respect to the infusion pump should be adjusted.

4. The method of claim 1, further comprising:
analyzing electrical signals representative of a pumping cycle associated with a cyclical pumping operation of the pump to move the fluid through the intravenous infusion line;
determining, based on the electrical signals, that that the pressure change did not result from relaxation of the intravenous infusion line,
wherein the motor speed or amount of compression is adjusted after the pressure change is determine to not result from relaxation of the intravenous infusion line.

5. The method of claim 4, wherein the electrical signals comprise time-varying signal levels, and wherein analyzing the electrical signals comprises:
determining a standardized signal level corresponding to the time-varying signal levels, wherein detecting the pressure change is outside of the pressure threshold comprises detecting the standardized signal level is outside the pressure threshold for a second threshold period of time.

6. The method of claim 5, wherein determining that that the pressure change did not result from relaxation of the intravenous infusion line comprises:
determining a decay of the standardized signal level;
adjusting the pressure threshold to correspond to the decay;
comparing the standardized signal level to the adjusted pressure threshold; and
confirming that the standardized signal level is outside the adjusted pressure threshold for a second period of time.

7. The method of claim 1,
wherein causing the infusion pump to adjust operation in accordance with the backpressure adjustment comprises adjusting a motor speed of the infusion pump or an amount of compression provided to the intravenous infusion line by the infusion pump based on a first algorithm, and wherein causing the infusion pump to adjust operation in accordance with the static pressure adjustment comprises adjusting a motor speed of the infusion pump or an amount of compression provided to the intravenous infusion line by the infusion pump based on a second algorithm.

8. The method of claim 1, after adjusting the motor speed of the infusion pump or the amount of compression provided to the intravenous infusion line:
obtaining, from a downstream pressure sensor of the infusion pump, a downstream pressure signal indicating that the pressure change remains outside of a pressure threshold; and
iteratively adjusting, based on a magnitude of the downstream pressure signal, the motor speed of the infusion pump or the amount of compression provided to the intravenous infusion line to cause the pressure change to be within the pressure threshold.

9. The method of claim 1, wherein the pressure change is due to static pressure and the amount of compression provided to the intravenous infusion line is adjusted by adjusting a pump finger compression distance.

10. The method of claim 1, further comprising:
obtaining, by the infusion pump before the pressure change is detected, parameters pertaining to a desired flow rate and a type of the intravenous infusion line;
wherein the motor speed of the infusion pump or an amount of compression provided to the intravenous infusion line is adjusted to adjust a current flow rate of the fluid within the intravenous infusion line towards the desired flow rate.

11. An infusion pump, comprising:
one or more pressure sensors;
a processor configured to:
detect, with the one or more pressure sensors, a pressure change outside of a pressure threshold in an intravenous infusion line associated with the infusion pump;
determine whether the pressure change is due to backpressure associated with a patient infusion line set associated with the intravenous infusion line or due to static pressure associated with a fluid container providing a fluid to the intravenous infusion line;
determine that the pressure change occurred over a first threshold period of time;
determine that the pressure change did not result from an occlusion event based on the pressure change occurring over the first threshold period of time; and
after determining that the pressure change did not result from an occlusion event:
when the pressure change is due to backpressure, provide an indication that the pressure change is associated with the patient infusion line set and adjusting a motor speed of the infusion pump or an amount of compression provided to the intravenous infusion line by the infusion pump based on a first algorithm; and
when the pressure change is due to static pressure, provide an indication that the pressure change is associated with the fluid container and adjusting the motor speed of the infusion pump or the amount of compression provided to the intravenous infusion line by the infusion pump based on a second algorithm.

12. The infusion pump of claim 11, wherein providing the indication that the pressure change is associated with the fluid container comprises providing an audio or visual indication that a height of the fluid container with respect to the infusion pump should be adjusted.

13. The infusion pump of claim 11, wherein providing the indication that the pressure change is associated with the patient infusion line set comprises providing an audio or visual indication that a height of the patient infusion line set with respect to the infusion pump should be adjusted.

14. The infusion pump of any claim 11, wherein the processor is further configured to:
analyze electrical signals representative of a pumping cycle associated with a cyclical pumping operation of the pump to move the fluid through the intravenous infusion line;
determine, based on the electrical signals, that that the pressure change did not result from relaxation of the intravenous infusion line,
wherein the motor speed or amount of compression is adjusted after the pressure change is determine to not result from relaxation of the intravenous infusion line.

15. The infusion pump of claim 14, wherein the electrical signals comprise time-varying signal levels, and wherein analyzing the electrical signals comprises:
determining a standardized signal level corresponding to the time-varying signal levels, wherein detecting the pressure change is outside of the pressure threshold comprises detecting the standardized signal level is outside the pressure threshold for a second threshold period of time.

16. The infusion pump of claim 15, wherein determining that that the pressure change did not result from relaxation of the intravenous infusion line comprises:
determining a decay of the standardized signal level;
adjusting the pressure threshold to correspond to the decay;
comparing the standardized signal level to the adjusted pressure threshold; and
confirming that the standardized signal level is outside the adjusted pressure threshold for a second period of time.

17. The infusion pump of claim 11, wherein the processor is further configured to:
determine that the pressure change is due to backpressure and static pressure;
adjust the motor speed of the infusion pump or the amount of compression provided to the intravenous infusion line based on the first and second algorithms.

18. The infusion pump of claim 1, wherein the processor is further configured to, after adjusting the motor speed of the infusion pump or the amount of compression provided to the intravenous infusion line:
obtain, from a downstream pressure sensor of the infusion pump, a downstream pressure signal indicating that the pressure change remains outside of a pressure threshold; and
iteratively adjust, based on a magnitude of the downstream pressure signal, the motor speed of the infusion pump or the amount of compression provided to the intravenous infusion line to cause the pressure change to be within the pressure threshold.

19. The infusion pump of claim 18, wherein the pressure change is due to static pressure and the amount of compression provided to the intravenous infusion line is adjusted by adjusting a pump finger compression distance.

20. The infusion pump of claim 11, wherein the processor is further configured to:
obtain, by the infusion pump before the pressure change is detected, parameters pertaining to a desired flow rate and a type of the intravenous infusion line,
wherein the motor speed of the infusion pump or an amount of compression provided to the intravenous infusion line is adjusted to adjust a current flow rate of the fluid within the intravenous infusion line towards the desired flow rate.
